(19) United States
(12) Patent Application Publication
(10) Pub. No.: US 2025/0366734 A1
(43) Pub. Date: Dec. 4, 2025

(54) Method and Apparatus for Human Gait Analysis

(71) Applicant: Orthini, LLC, Portland, OR (US)

(72) Inventors: Michael David RIES, Reno, NV (US); Michael COATES, Portland, OR (US); LaJean Ruth Lawson, Portland, OR (US); Christopher J. Nanson, Sherwood, OR (US); Kurt Richard GRITMAN, Portland, OR (US)

(21) Appl. No.: 18/731,079

(22) Filed: May 31, 2024

(51) Int. Cl. A61B 5/103; G16H 40/67

(52) U.S. Cl. CPC A61B 5/1038; G16H 40/67

(57) ABSTRACT

An electronic computing system to generate information relating to a gait of a human. A first sensor proximate the heel detects a first location of a heel of the human at an initiation of a swing phase for a corresponding leg. A second sensor proximate the knee detects a first, concurrent, location of a corresponding knee. The system calculates a first angle θ₁ defined by a vertex, A, at the first location of the knee formed by a first side, B, defined by a straight vertical line that intersects the first location of the knee and a second side, C, defined by a straight line that intersects the first location of the knee and the first location of the heel. The system then calculates a maximum flexion of the knee at the initiation of the swing phase for the corresponding leg based on the first angle θ₁.

### TECHNICAL FIELD

Embodiments of the invention are related to gait analysis, for example, in humans.

### BACKGROUND

Gait is the pattern of how an animal moves its body. A human's gait is the pattern of how a person walks. Many different diseases and conditions can affect a person's gait and point or lead to problems with walking. For example, after surgery, such as knee surgery, knee replacement surgery, or knee arthroplasty, or when experiencing gait impairment after treatment for conditions such as osteoarthritis, stroke, or diabetes, analysis of the patient's gait can indicate how recovery is proceeding after surgery or treatment. Alternatively, a person, such as an athlete, might participate in biomechanical analysis, including gait analysis, to improve their performance in an activity or sport such a walking, running, hiking, cross-country skiing, etc. Typically, such analysis is conducted in a doctor-patient setting or other type of service provider setting, for example, in a laboratory, hospital or rehabilitation center, under the doctor's or other professional's supervision. It would be helpful to have the ability to conduct this analysis outside a laboratory setting, for example, in the morning when the person experiencing gait impairment first puts weight on their legs, or when the person experiments with or changes one or more of cadence, velocity, distance, acceleration, stride length, shoes, wearing a weight-bearing article such as a rucksack, terrain or slope in a selected activity, with or without a doctor's or professional's supervision. Gait analysis in a controlled laboratory setting may not reflect gait patterns that occur during in vivo daily activities such as walking or climbing on uneven surfaces, during participation in sports or recreational activities, or at various points of muscle fatigue that may occur over time. Gait analysis with use of musculoskeletal monitoring devices during daily activities may provide more clinically relevant gait analysis than what can be obtained in a laboratory setting.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 is a flowchart for calculating a maximum flexion of a knee during a swing phase of a human's gait according to embodiments of the invention;

FIG. 2 is a flowchart for calculating maximum flexions of a knee over consecutive swing phases of a human's gait according to embodiments of the invention;

FIG. 3 is a flowchart for calculating a minimum flexion of a knee during a swing phase of a human's gait and identifying excursion of the knee according to embodiments of the invention;

FIG. 4 is a flowchart for calculating a maximum extension (minimum flexion) of a knee during a stance phase of a human's gait according to embodiments of the invention;

FIG. 5A is a flowchart for determining a health status of a knee according to embodiments of the invention;

FIG. 5B is a flowchart for determining a health status of a knee according to embodiments of the invention;

FIG. 6 is a flowchart for calculating a maximum flexion of a knee during a stance phase of a human's gait according to embodiments of the invention;

FIG. 7A is a flowchart for determining a health status of a knee according to embodiments of the invention;

FIG. 7B is a flowchart for determining a health status of a knee according to embodiments of the invention;

FIG. 8 is a flowchart for calculating the length of a stance phase of a human's gait according to embodiments of the invention;

FIG. 9 is a diagram of a swing phase of a human's gait according to embodiments of the invention;

FIG. 10 is a diagram of a stance phase of a human's gait according to embodiments of the invention;

FIG. 11A depicts the geometry involved in determining maximum flexion of the knee at the initiation of a swing phase of the corresponding leg according to an embodiment of the invention;

FIG. 11B depicts the geometry involved in determining the minimum flexion of the knee at the termination of the swing phase of the corresponding leg according to an embodiment of the invention;

FIGS. 12A and 12B depict knee flexion excursion from the initiation of the swing phase to the termination of the swing phase for the corresponding leg according to an embodiment of the invention;

FIGS. 13A and 13B depict stride length due to motion at the hip, including both hip extension stride length and hip flexion stride length, during the swing phase for the corresponding leg according to an embodiment of the invention;

FIGS. 14A, 14B and 14C depict hip stride length due to motion at the hip, including both hip extension stride length and hip flexion stride length, as measured by an accelerometer placed at the knee, during the swing phase for the corresponding leg, according to an embodiment of the invention

FIGS. 15A and 15B depict stride length due to knee motion, including both knee flexion stride length and knee extension stride length, during the swing phase for the corresponding leg according to an embodiment of the invention;

FIGS. 16A and 16B depict a calculation for maximum knee flexion angle based on knee flexion stride length, during the swing phase for the corresponding leg according to an embodiment of the invention;

FIGS. 17A and 17B depict a calculation for the horizontal distance that the body moves from maximum knee flexion angle based on knee flexion stride length, during the swing phase for the corresponding leg according to an embodiment of the invention;

FIGS. 18A and 18B depict a calculation for the minimum knee flexion (maximum knee extension) angle based on knee extension stride length, during the swing phase for the corresponding leg according to an embodiment of the invention;

FIGS. 19A and 19B depict a calculation for the horizontal distance that the body moves from minimum knee flexion (maximum knee extension) angle based on knee extension stride length, during the swing phase for the corresponding leg according to an embodiment of the invention;

FIG. 20 depicts the knee excursion angle from stride length due to knee motion, including the maximum knee flexion angle and the minimum knee flexion angle, during the swing phase for the corresponding leg according to an embodiment of the invention;

FIG. 21 depicts the forward movement of the upper body due to knee stride length, according to an embodiment of the invention.

### DETAILED DESCRIPTION

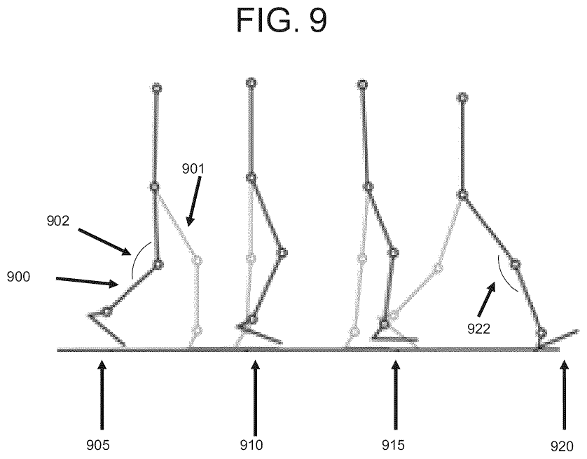

When a human walks, each leg cycles through a stance phase followed by a swing phase of the human's gait. While one leg is in contact with a surface such as the ground or a floor during that leg's stance phase, the other leg is not in contact with the surface and contemporaneously swings forward during the other leg's corresponding swing phase. FIG. 9 illustrates the swing phase for a human's leg 900 (e.g., right leg), while the other leg 901 (e.g., left leg) is in the stance phase. At 905, the swing phase for leg 900 is initiated by lifting the corresponding heel, followed by the ball, and then the toes, of the foot of leg 900 so that the heel and then finally the toes of the foot of leg 900 break contact with the surface. Concurrently, the knee of leg 900 is at maximum flexion as depicted at 902. At 910 and 915, the leg is depicted at various points in the middle of its swing phase, with the knee leading the foot. Later in the swing phase, the foot is leading the knee, and at the termination or end of the swing phase at 920, the heel of the foot is further ahead of the knee when it contacts the surface.

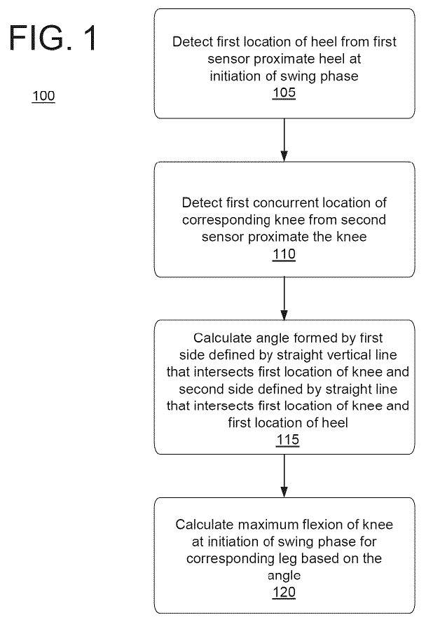

With reference to FIGS. 1 and 9, embodiments of the invention include a computer-implemented method 100 for an electronic computing system working in conjunction with sensors to determine the maximum flexion of the knee, depicted at 902, at toe off or the initiation of the swing phase 905 for leg 900. The electronic computing system has a memory to store instructions and a processor to execute the instructions to generate information relating to a gait of a human, such as information relating to the maximum flexion of the knee at the initiation of the swing phase for the corresponding leg. The process begins at step 105 by detecting a first, or initial, location of the heel of the human from a first sensor coupled in communication with the electronic computing system and proximate the heel at the initiation of the swing phase 905 for leg 900. The first sensor may be strapped or affixed, for example, via adhesive, to the heel or may be attached or embedded in an article worn on or around the foot, such as a wrap, a sock, or a shoe. It is appreciated that the initiation of the swing phase 905 for leg 900 coincides with or immediately follows termination of a stance phase for leg 900.

The process continues at step 110 by detecting a first, concurrent, location of the corresponding knee from a second sensor coupled in communication with the electronic computing system and proximate the knee. The second sensor may be strapped or affixed, for example, via adhesive, to the knee or may be attached or embedded in an article worn on or about the knee, such as a wrap, a bandage, kinesiology tape, a brace, clothing, etc.

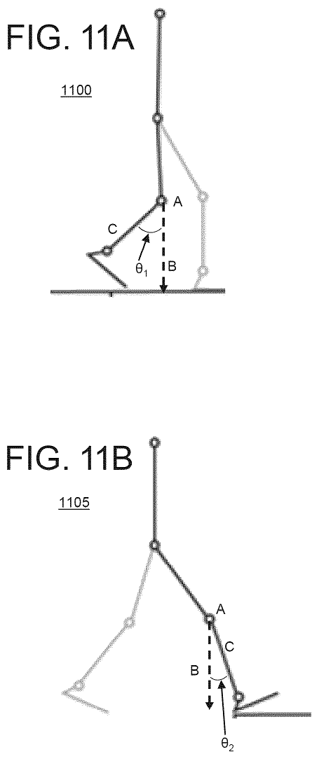

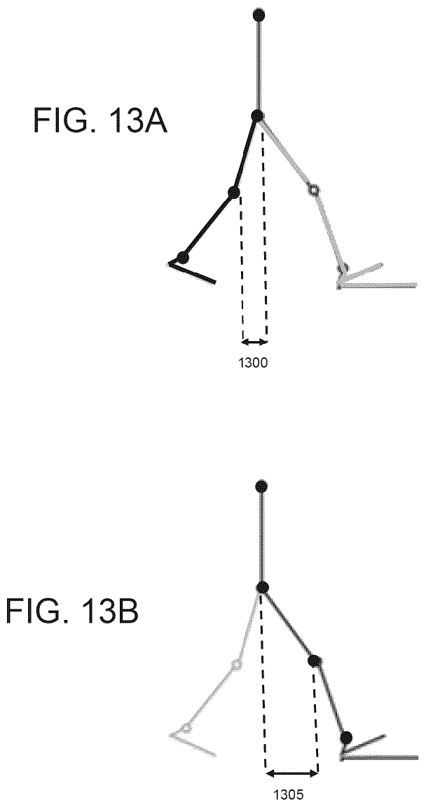

With further reference to the depiction 1100 in FIG. 11A, the process continues at step 115 by calculating a first angle θ₁ defined by a vertex, A, positioned at the first location of the knee formed by a first side, B, defined by a straight line that intersects the first location of the knee and a second side, C, defined by a straight line that intersects the first location of the knee and the first location of the heel. While the illustration depicts the upper portion of the leg, i.e., the femur, in a vertical position, it is appreciated that the femur could be in a different position, for example, as depicted in FIGS. 13A-17B. The process then calculates at 120 a maximum flexion of the knee at the initiation of the swing phase for the corresponding leg based on the first angle θ₁. For example, the maximum flexion of the knee at the initiation of the swing phase may be calculated as one of two supplementary angles the sum of which is 180 degrees. In this instance, assuming the femur is generally vertical at the initiation of the swing phase, subtracting the first angle θ₁ from 180 degrees yields an angle corresponding to the maximum flexion of the knee. For example, if the first angle θ₁ is 30 degrees, the maximum flexion of the knee would be 150 degrees. In other instances where the femur is not generally in a vertical position, a sensor, such as an accelerometer, can determine the angle of the femur, for example, with respect to a vertical axis, at the initiation of the swing phase, and the embodiment may then calculate the maximum flexion of the knee based on the first angle θ₁ and the angle of the femur.

According to an embodiment, the process of detecting the first location of the heel of the human from the first sensor at the initiation of the swing phase involves detecting the first location of the heel from the first sensor based on the heel breaking contact with a surface as detected by the first sensor. The first sensor may, for example, detect the absence of pressure on the bottom of the heel when the heel breaks contact with the surface.

In another embodiment, the initiation of the swing phase for the leg may be based on when the ball of the foot, or a toe (e.g., the innermost toe, also referred to as the hallux or “big toe”) or toes of the foot, break contact with the surface. (Reference hereinafter to toes or toes sensor is also meant to include an individual toe and corresponding toe sensor, whether the hallux or second (“index”) toe, or another toe, and corresponding toe sensor). In such case, the process at step 105 detects a first, or initial, location of the ball or toes of the foot from a first sensor coupled in communication with the electronic computing system and proximate the ball or toes of the foot at the initiation of the swing phase 905 for leg 900. In another embodiment, multiple sensors may be deployed at two or more of the heel, ball and toes of the foot, and some combination of the respective locations and timings of the heel, ball, and toes of the foot breaking contact and the associated amount of pressure or absence thereof between the heel, ball, or toes of the foot and the surface may be considered in defining the initiation of the swing phase for the corresponding leg.

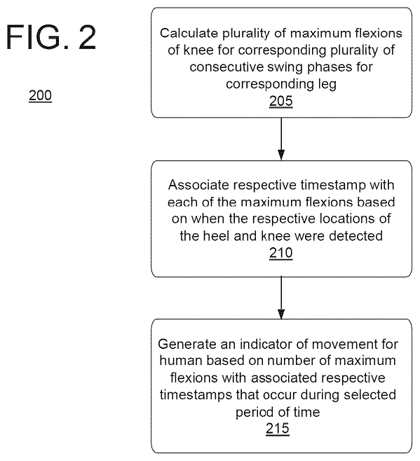

With reference to FIGS. 1 and 2, according to an embodiment 200, repeating steps 105-120 as a person walks, allows for calculating at step 205 any number of consecutive maximum flexions of the knee for a corresponding number of consecutive initiations of the swing phase for the corresponding leg. At step 210, the process associates a respective timestamp with each of the consecutive maximum flexions based on the concurrent detections of the respective first locations of the heel and knee. Thus, a history or series of maximum flexions of the knee for a corresponding number of consecutive initiations of the swing phase for the corresponding leg can be generated over a period of time. The electronic computing system can then at step 215 generate one or more indicators of movement for the human based on this data. For example, given each maximum flexion of the knee coincides with the initiation of the swing phase for the corresponding leg, and given the location of and time at which the heel breaks contact with the surface is detected for each initiation, it is possible to determine stride-length for the leg between any two consecutive swing phases as the distance between the locations of the heel at two consecutive swing phases of the leg. Furthermore, given each maximum flexion of the knee coincides with the time at which the heel breaks contact with the surface for each initiation, it is possible to determine cadence, i.e., the number of steps per unit of time. Furthermore, given stride-length and cadence, it is possible to determine velocity, and acceleration for a selected period of time.

It is appreciated that the above-described embodiments, as well as the embodiments described below, may be performed in real-time, in which data detected by the sensors are relayed immediately to the electronic computing system via a wired or wireless communication medium and the calculations performed by the electronic computing system. The electronic computing system may likewise generate output in real-time to a display device or display space coupled in communication with the electronic computing system for viewing by the human and/or a service provider for immediate, real-time feedback. Alternatively, or additionally, the described embodiments may be performed in batch mode, in which data detected by the sensors are relayed or uploaded periodically or upon user input requesting a relay or upload, for example, after one or more sessions of selected activity are completed, to the electronic computing system via a wired or wireless communication medium. The electronic computing system then performs the calculations and generates output to the display device or display space for viewing by the human. The electronic computing system may likewise generate output in real-time to a display device or display space coupled in communication with the electronic computing system for viewing by the human and/or the service provider for viewing.

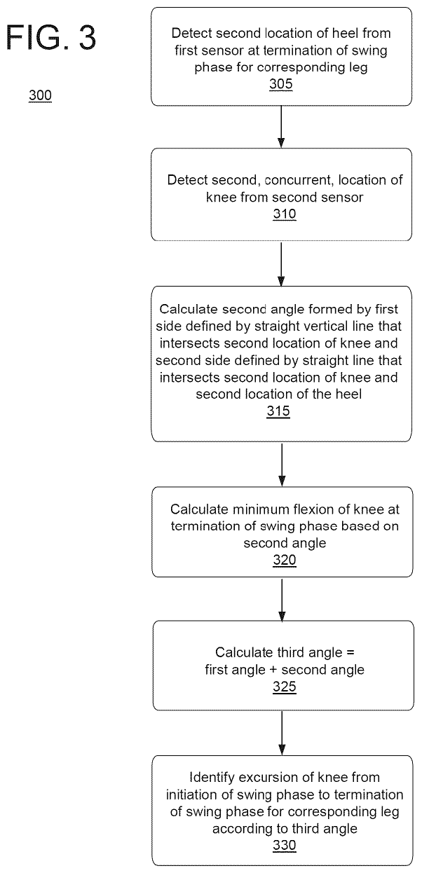

With reference to FIGS. 3 and 9, embodiments of the invention include a computer-implemented method 300 for an electronic computing system working in conjunction with sensors to determine the minimum flexion (or a maximum extension) of the knee, depicted at 922, at the termination of the swing phase 920 for leg 900. The process begins at step 305 by detecting a second location of the heel from the first sensor at the termination of the swing phase 920 for leg 900. It is appreciated that the termination of the swing phase 920 for leg 900 coincides with or immediately precedes initiation of a subsequent stance phase for leg 900.

The process continues at step 310 by detecting a second, concurrent, location of the corresponding knee from the second sensor coupled in communication with the electronic computing system and proximate the knee.

With further reference to the depiction 1105 in FIG. 11B, the process continues at step 315 by calculating a second angle θ₂ defined by a vertex, A, positioned at the second location of the knee formed by a first side, B, defined by a straight vertical line that intersects the second location of the knee and a second side, C, defined by a straight line that intersects the second location of the knee and the second location of the heel. The process then calculates at step 320 a minimum flexion (or maximum extension) 922 of the knee at the termination of the swing phase for the corresponding leg based on the second angle θ₂.

According to an embodiment, the process of detecting at 305 the second location of the heel of the human from the first sensor at the termination of the swing phase involves detecting the second location of the heel from the first sensor based on when the heel contacts a surface as detected by the first sensor. The first sensor may, for example, detect pressure on the bottom of the heel when the heel contacts the surface.

In another embodiment, the termination of the swing phase for the leg may be based on when the ball of the foot, or the toes of the foot, contact the surface. In such case, the process at step 305 detects the second location of the ball or toes of the foot from the first sensor coupled in communication with the electronic computing system and proximate the ball or toes of the foot at the termination of the swing phase 920 for leg 900. In another embodiment, multiple sensors may be deployed at two or more of the heel, ball and toes of the foot, and some combination of the respective locations and timings of the heel, ball, and toes of the foot contacting the surface may be considered in defining the termination of the swing phase for the corresponding leg.

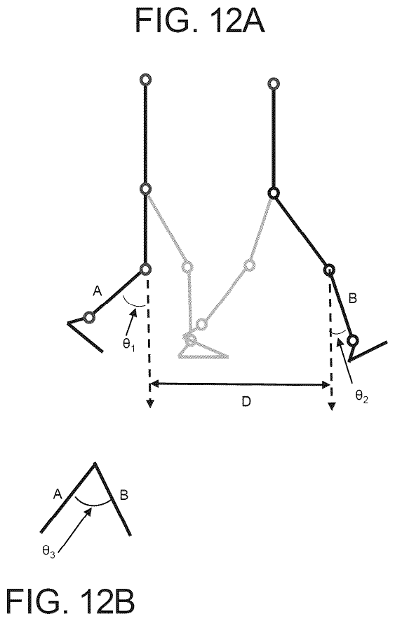

Further with reference to FIGS. 3, 11A, 11B, 12A and 12B, given the first angle θ₁ is calculated at step 115 at the initiation of the swing phase 905 for leg 900, and given the second angle θ₂ is calculated at step 315 at the termination of the swing phase 920 for leg 900, the process can then readily calculate at step 325 a third angle θ₃ based on the first angle and the second angle. For example, the third angle may be calculated as the sum of the first and second angles. The process can then, at step 330, generate an indicator of a knee flexion excursion from the initiation of the swing phase 905 to the termination of the swing phase 920 for the corresponding leg 900 as or according to the third angle θ₃.

With reference to FIGS. 13A-21, various calculations associated with stride length and gait analysis according to embodiments of the invention are described below.

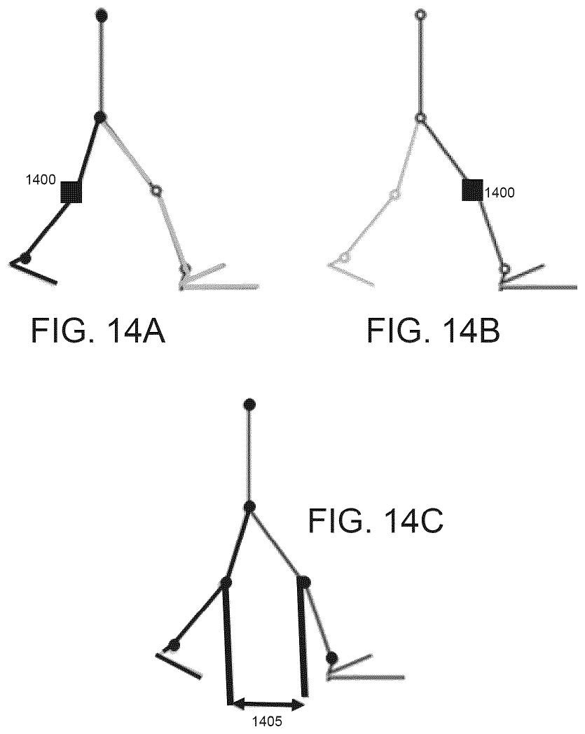

FIGS. 13A and 13B depict stride length due to motion at the hip, including both hip extension stride length 1300 depicted in FIG. 13A and hip flexion stride length 1305 depicted in FIG. 13B, during the swing phase for the corresponding leg according to an embodiment of the invention. FIGS. 14A, 14B and 14C depict an accelerometer 1400 placed at the knee to measure the hip stride length 1405 due to motion at the hip, including both hip extension stride length 1300 and hip flexion stride length 1305, during the swing phase for the corresponding leg, according to an embodiment of the invention.

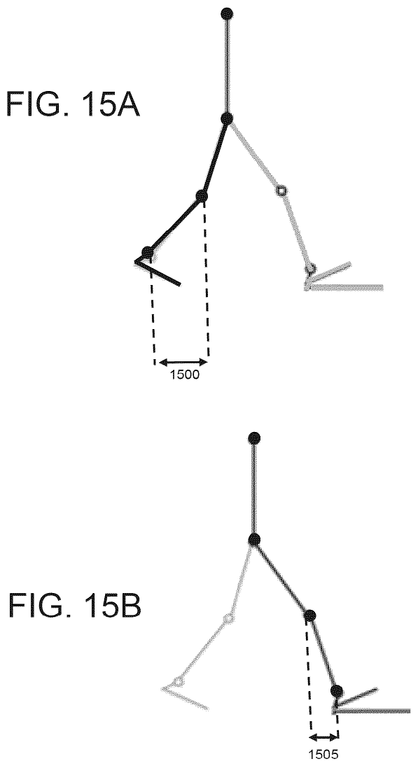

FIGS. 15A and 15B depict the corresponding portion of stride length due to knee motion, including both knee flexion stride length 1500 depicted in FIG. 15A, and knee extension stride length 1505 depicted in FIG. 15B, during the swing phase for the corresponding leg according to an embodiment of the invention.

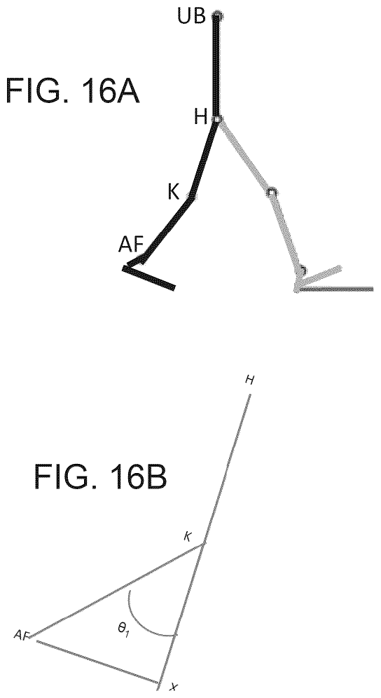

FIGS. 16A and 16B depict a calculation for maximum knee flexion angle based on knee flexion stride length 1500, during the swing phase for the corresponding leg according to an embodiment of the invention. In FIGS. 16A and 16B, UB references the top of the upper body of a human, H references the human's hip, K references the knee of a corresponding leg of the human during a swing phase, AF references the ankle in flexion for the corresponding leg, the line HK defines the length and orientation of the femur for the corresponding leg, the line KAF defines the length and orientation of the tibia in flexion for the corresponding leg, and the angle θ₁ is a measure of the maximum knee flexion angle between the femur and the tibia in flexion for the corresponding leg. Appropriate sensors placed in at least two appropriate locations among the ankle, shank, knee, femur and hip capture the necessary data for an electronic computing system in communication, and working in conjunction, with the sensors to determine the maximum knee flexion angle.

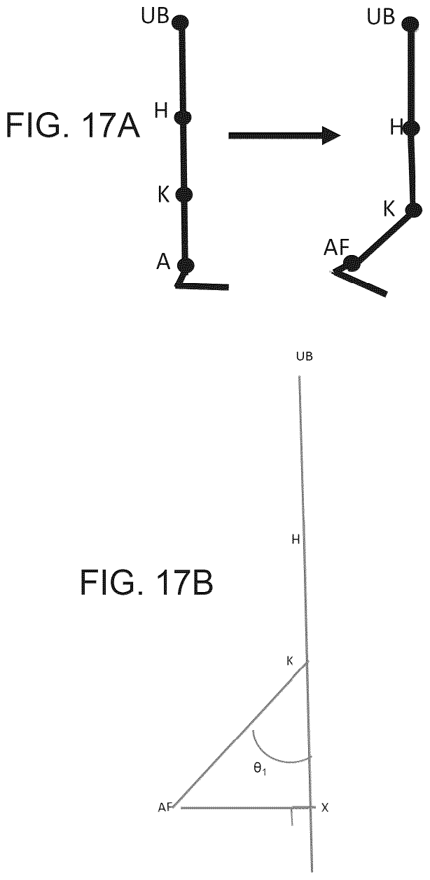

FIGS. 17A and 17B depict a calculation for the horizontal distance that the body moves from a first position in which the entire leg is essentially in a vertical orientation to a second position in which the leg is at the maximum knee flexion angle based on knee flexion stride length, during the end of the stand phase and the initiation of the swing phase for the corresponding leg according to an embodiment of the invention. In FIGS. 17A and 17B, UB references the top of the upper body of a human, H references the human's hip, K references the knee of a corresponding leg of the human during a swing phase, A references the ankle in a static, neutral position, neither in extension nor flexion, for the corresponding leg, AF references the ankle in flexion for the corresponding leg, the line HK defines the length and orientation of the femur for the corresponding leg, the line KAF defines the length and orientation of the tibia in flexion for the corresponding leg, the angle θ₁ is a measure of the maximum knee flexion angle between the femur and the tibia in flexion for the corresponding leg, and the line AFX defines the horizontal distance the upper body moves due to maximum knee flexion of the corresponding leg during the swing phase of the human's gait. The line AFX can be calculated for the right triangle given the length of the hypotenuse and the three angles that make up the interior of the right triangle are known or easily calculated. Appropriate sensors placed in at least two appropriate locations among the ankle, shank, knee, femur and hip capture the necessary data for an electronic computing system in communication, and working in conjunction, with the sensors to determine the maximum knee flexion angle.

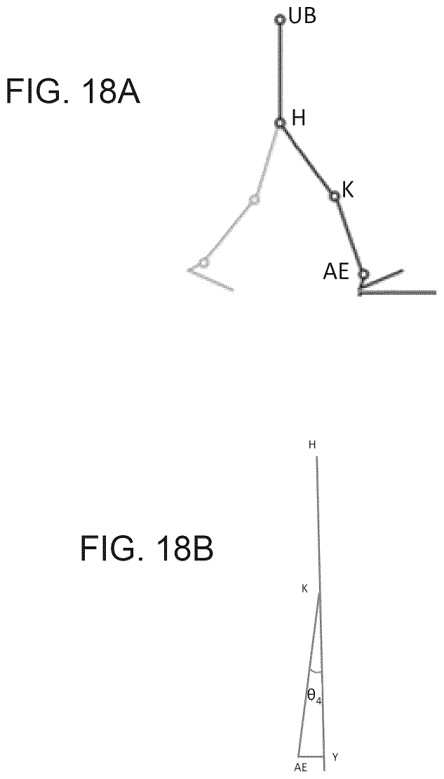

FIGS. 18A and 18B depict a calculation for the minimum knee flexion (maximum knee extension) angle based on knee extension stride length, during the swing phase for the corresponding leg according to an embodiment of the invention. In FIGS. 18A and 18B, UB references the top of the upper body of a human, H references the human's hip, K references the knee of a corresponding leg of the human during a swing phase, AE references the ankle in extension for the corresponding leg, the line HK defines the length and orientation of the femur for the corresponding leg, the line KAE defines the length and orientation of the tibia in extension for the corresponding leg, and the angle θ₄ is a measure of the minimum knee flexion angle between the femur and the tibia in extension for the corresponding leg. Appropriate sensors placed in at least two appropriate locations among the ankle, shank, knee, femur and hip capture the necessary data for an electronic computing system in communication, and working in conjunction, with the sensors to determine the minimum knee flexion angle.

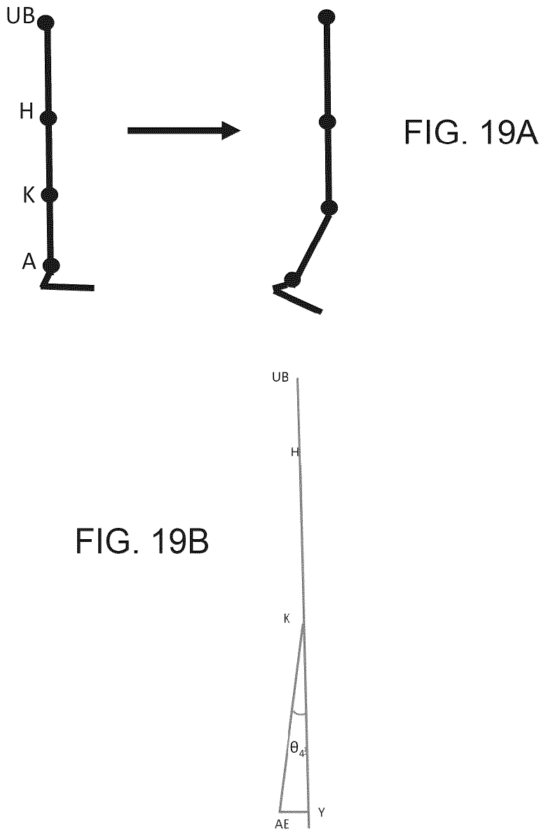

FIGS. 19A and 19B depict a calculation for the horizontal distance that the body moves from minimum knee flexion (maximum knee extension) angle based on knee extension stride length, during the termination of the swing phase for the corresponding leg according to an embodiment of the invention. In FIGS. 19A and 19B, UB references the top of the upper body of a human, H references the human's hip, K references the knee of a corresponding leg of the human during a swing phase, A references the ankle in a static, neutral position, neither in extension nor flexion, for the corresponding leg, AE references the ankle in extension for the corresponding leg, the line HK defines the length and orientation of the femur for the corresponding leg, the line KAE defines the length and orientation of the tibia in minimum flexion for the corresponding leg, the angle θ₄ is a measure of the minimum knee flexion angle between the femur and the tibia in extension for the corresponding leg, and the line AEY defines the horizontal distance the upper body moves due to minimum knee flexion of the corresponding leg during the swing phase of the human's gait. The line AEY can be calculated for the right triangle given the length of the hypotenuse and the three angles that make up the interior of the right triangle are known or easily calculated. Appropriate sensors placed in at least two appropriate locations among the ankle, shank, knee, femur and hip capture the necessary data for an electronic computing system in communication, and working in conjunction, with the sensors to determine the maximum knee flexion angle.

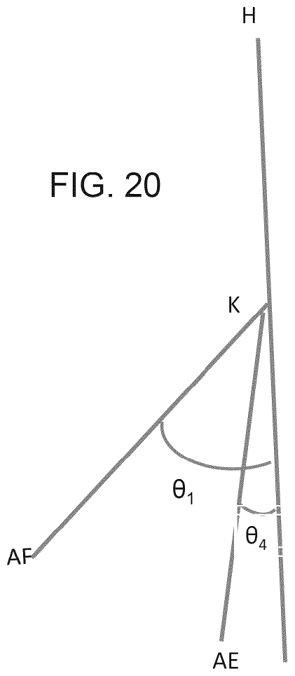

FIG. 20 depicts the knee flexion excursion from the initiation of the swing phase to the termination of the swing phase for the corresponding leg from stride length due to knee motion, including the maximum knee flexion angle θ₁ (as depicted and calculated with reference to FIGS. 16A, 16B, 17A and 17B above) and the minimum knee flexion angle θ₄ (as depicted and calculated with reference to FIGS. 18A, 18B, 19A and 19B above).

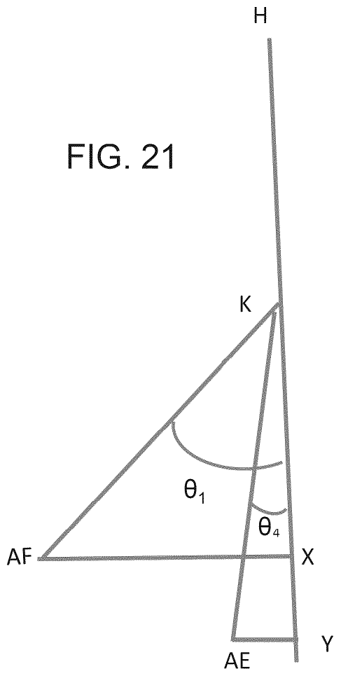

FIG. 21 depicts the forward movement of the upper body due to knee stride length, according to an embodiment of the invention. All the references in the figure are as defined above in FIGS. 16A-20. Notably, forward movement of the upper body is readily determined given the lengths of lines AFX and AEY by simply subtracting the latter (AEY) from the former (AFX). Thus, forward movement of the upper body due to knee stride length is equal to the length of line AFX—the length of line AEY.

Sin (θ¹)=length of line AFX divided by length of KAF, and sin (θ₄)=length of line AEY divided by length of line KAE.

From the above, knee stride length=length of line KAF (sin (θ₁)−length of line KAE (sin (θ₄). Thus, knee stride length=(length of the tibia (sin (maximum knee flexion angle))−((length of tibia (sin (minimum knee flexion angle)).

From the above:

$$total stride length = hip stride length + knee stride length ;$$

$$knee cadence = knee stride length / knee stride time ;$$

$$hip cadence - hip stride length / hip stride time ; and$$

$$cadence = total stride length / total stride time .$$

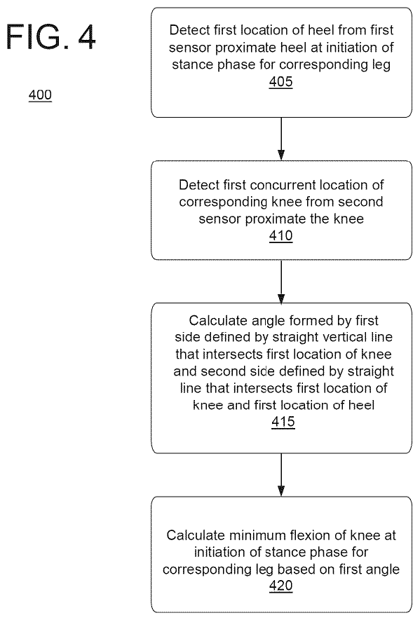

The above discussed embodiments relate to methods of determining various metrics regarding a human's gait during the swing phase of the human's gait, with reference to FIGS. 1-3 and 9. The below discussed embodiments relate to methods for determining various metrics regarding a human's gait during the stance phase of the human's gait, with reference to FIGS. 4-8 and 10. The following embodiments involve obtaining simultaneous or concurrent knee flexion data and foot pressure data, such as foot contact detected by one or more of a heel sensor, a ball sensor and a toe or toes sensor, at the beginning or initiation of a stance phase of a human's gait, at a middle of the stance phase where a generally vertical tibia is detected by the heel sensor and the ball sensor owing to pressures detected by the heel and ball sensors being roughly the same, and at the end of the stance phase, when the heel, ball, or toes lifting off the surface is detected by the heel, the ball, or the toes sensor. The general concept underlying the disclosed embodiments is to combine foot pressure data with knee flexion data during gait. A most efficient gait involves maximum heel pressure at maximum knee extension (heel strike) and maximum toe pressure at maximum knee flexion (toe off). For example, after total knee replacement arthroplasty surgery (TKA) or other surgery involving the knee, ankle, heel, toes, etc., or after injury to one or more of the same, the degree to which heel strike and toe off pressure data is synchronized with knee flexion during gait can be used as a measure of gait efficiency. Tracking this data over time, and/or contrasting this data with tracking similar data for the human's other leg, allows for monitoring gait impairment, such as during recovery from post-op knee replacement, while undertaking a selected activity such as walking, climbing a hill, running, skiing, or everyday tasks, such as moving around one's house, climbing or descending stairs, checking the mailbox located at the end of a driveway or block for mail, mowing the lawn, and grocery shopping.

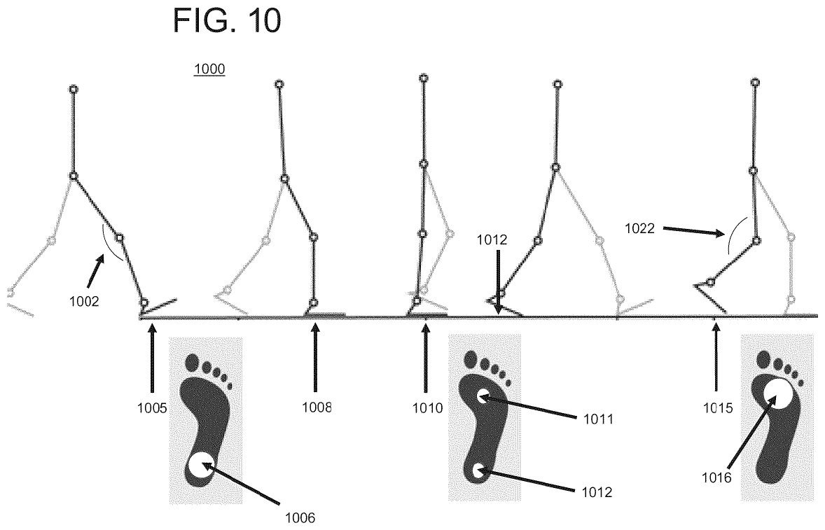

With reference to FIGS. 4 and 10, embodiments of the invention include a computer-implemented method 400 for an electronic computing system working in conjunction with sensors to determine the minimum flexion (or a maximum extension) of the knee, depicted at 1002, at the initiation of the stance phase 1005 for leg 900. The process begins at step 405 by detecting a first location of the heel for that leg from the first sensor at the initiation of the stance phase 1005 for leg 900. It is appreciated that the initiation of the stance phase 1005 for leg 900 coincides with or immediately follows termination of a previous swing phase for leg 900.

The process continues at step 410 by detecting a first, concurrent, location of the corresponding knee from the second sensor coupled in communication with the electronic computing system and proximate the knee.

With reference to FIG. 11A, the process continues at step 415 by calculating a first angle θ₁ defined by a vertex, A, positioned at the first location of the knee formed by a first side, B, defined by a straight vertical line that intersects the first location of the knee and a second side, C, defined by a straight line that intersects the first location of the knee and the first location of the heel. The process then calculates at step 420 a minimum flexion (or maximum extension) 1002 of the knee at the initiation of the stance phase for the corresponding leg based on the first angle θ₁.

According to an embodiment, the process of detecting at 405 the first location of the heel of the human from the first sensor at the initiation of the stance phase involves detecting the first location of the heel from the first sensor based on the heel contacting a surface as detected by the first sensor. The first sensor, may, for example, detect significant or a threshold amount of pressure (depicted by the relatively large dot 1006) at or on the bottom of the heel when the heel makes contact with the surface.

In another embodiment, the initiation of the stance phase for the leg may be based on when the ball of the foot, or the toes of the foot, makes contact with the surface. In such case, the process at step 405 detects the first location of the ball or toes of the foot from a respective sensor proximate the ball or toes of the foot and coupled in communication with the electronic computing system at the initiation of the stance phase 1005 for leg 900. In another embodiment, multiple sensors may be deployed at two or more of the heel, ball and toes of the foot, and some combination of the respective locations and timings of the heel, ball, and toes of the foot contacting the surface may be considered in defining the initiation of the stance phase for the corresponding leg.

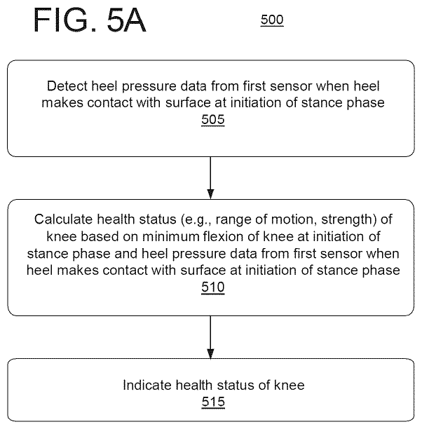

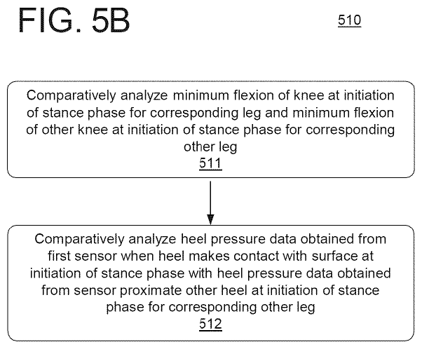

With reference to FIGS. 5A, 5B and 10, embodiments include a computer-implemented method 500 for an electronic computing system working in conjunction with sensors to detect at step 505 heel pressure data from the first sensor based on the heel contacting a surface at the initiation of the stance phase. Conversely or additionally, the step may involve detecting a lack of ball pressure from ball pressure data detected by a sensor proximate a ball of the foot or detecting a lack of toe pressure data detected by another sensor proximate a toe or toes of the foot, given no contact between the ball or toes of the foot and the surface at the initiation of the stance phase.

The process continues at step 510 by calculating a health status of the knee based on the calculated minimum flexion of the knee at the initiation of the stance phase and the heel pressure data from the first sensor based on the heel contacting the surface at the initiation of the stance phase (e.g., a range of motion based on the minimum flexion/maximum extension of the knee, and a measure of strength of or pain tolerance associated with the knee or other joint of the leg based on a correlation with the amount of pressure detected by one or more of the heel, ball, and toes sensors). According to some embodiments, the process can then generate at step 515 an indicator of the health status of the knee in response to the calculated health status of the knee.

Regarding step 510, with reference to FIGS. 5A and 5B, calculating the health status of the knee based on the calculated minimum flexion of the knee at the initiation of the stance phase and the heel pressure data from the first sensor based on the heel making contact with the surface at the initiation of the stance phase involves, according to one embodiment, at step 511, comparatively analyzing the calculated minimum flexion of the knee at the initiation of the stance phase for the corresponding leg with a likewise or similarly calculated minimum flexion of the other knee of the human at the initiation of the stance phase for the human's other leg, and at step 512, comparatively analyzing the heel pressure data detected by the first sensor based on the heel contacting with the surface at the initiation of the stance phase with heel pressure data detected by a sensor proximate the other heel of the human at the initiation of the stance phase for the corresponding other leg. This analysis may be particularly helpful, for example, when one knee is recovering from surgery or impairment and the other knee, the “good knee”, is generally functioning normally and pain free. Over time, e.g., a matter of hours, days, weeks, months, the comparative analysis may offer an overall indication of the health status, or a change in the health status, of the impaired knee relative to the so-called good knee.

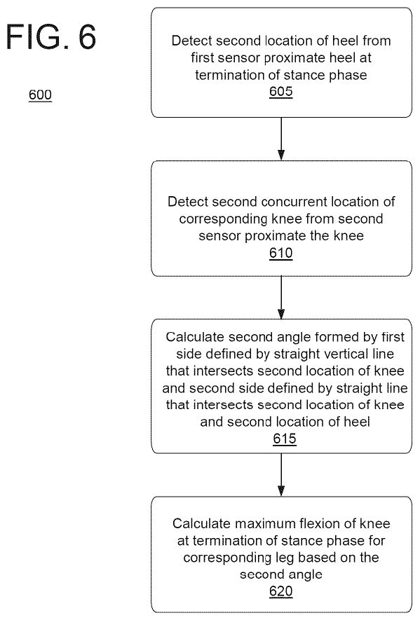

With reference to FIGS. 6 and 10, at 1015, the stance phase for leg 900 is terminated by lifting the corresponding heel, followed by the ball, and then the toes of the foot of leg 900 so that the heel and then finally the toes break contact with the surface. Concurrently, the knee of leg 900 is at maximum flexion as depicted at 922.

Embodiments of the invention include a computer-implemented method 600 for an electronic computing system working in conjunction with sensors to determine the maximum flexion of the knee, depicted at 1022, at the termination of the stance phase 1015 for leg 900. The electronic computing system has a memory to store instructions and a processor to execute the instructions to generate information relating to a gait of a human, such as information relating to the maximum flexion of the knee at the termination of the stance phase for the corresponding leg. The process begins at step 605 by detecting a second location of the heel of the human from a first sensor coupled in communication with the electronic computing system and proximate the heel at the termination of the stance phase 1015 for leg 900. It is appreciated that the termination of the stance phase 1015 for leg 900 coincides with or immediately precedes initiation of a swing phase for leg 900.

The process continues at step 610 by detecting a second, concurrent, location of the corresponding knee from a second sensor coupled in communication with the electronic computing system and proximate the knee.

The process continues at step 615 by calculating a second angle θ₂ defined by a vertex, A, positioned at the second location of the knee formed by a first side, B, defined by a straight vertical line that intersects the second location of the knee and a second side, C, defined by a straight line that intersects the second location of the knee and the second location of the heel. The process then calculates at 620 a maximum flexion of the knee at the termination of the stance phase for the corresponding leg based on the second angle θ₂. For example, the maximum flexion of the knee at the termination of the stance phase may be calculated as one of two supplementary angles the sum of which is 180 degrees. In this instance, assuming the femur is generally vertical at the termination of the stance phase, subtracting the second angle θ₂ from 180 degrees yields an angle corresponding to the maximum flexion of the knee.

According to an embodiment, the process of detecting the second location of the heel of the human from the first sensor at the termination of the stance phase involves detecting the second location of the heel from the first sensor based on the heel breaking contact with a surface as detected by the first sensor. The first sensor may, for example, detect the absence of pressure at or on the bottom of the heel when the heel breaks contact with the surface.

In another embodiment, the termination of the stance phase for the leg may be based on when the ball of the foot, or a toe (e.g., the “big toe”) or toes of the foot, break contact with the surface. In such case, the process at step 605 detects the second location of the ball or toes of the foot from a sensor coupled in communication with the electronic computing system and proximate the ball or toes of the foot at the termination of the stance phase 1015 for leg 900. In such an embodiment, once the heel is lifted at termination of the stance phase at 1015, the ball sensor may, for example, detect a significant or a threshold amount of pressure (depicted by the relatively large dot 1006) at or on the ball of the foot when the heel breaks contact with the surface. In another embodiment, multiple sensors may be deployed at two or more of the heel, ball and toes of the foot, and some combination of the respective locations and timings of the heel, ball, and toes of the foot breaking contact and the associated amount of pressure or absence thereof between the heel, ball, or toes of the foot and the surface may be considered in defining the termination of the stance phase for the corresponding leg.

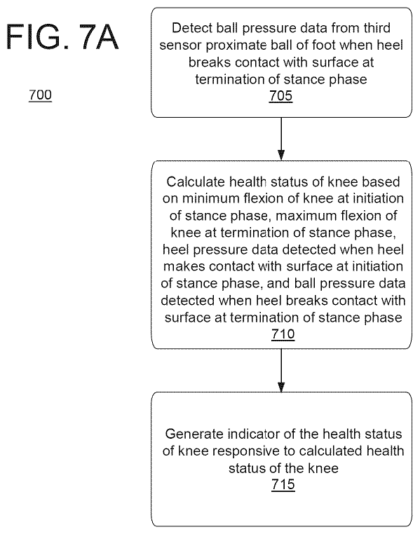

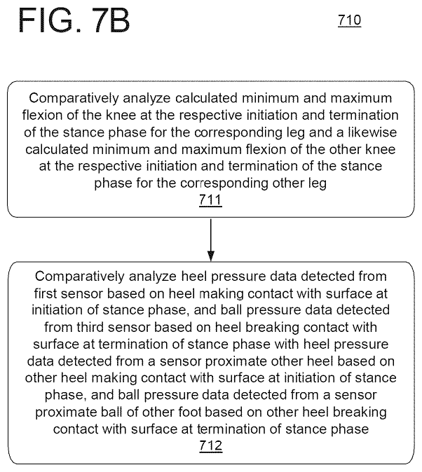

With reference to FIGS. 7A, 7B and 10, embodiments include a computer-implemented method 700 for an electronic computing system working in conjunction with sensors to detect at step 705 ball pressure data from the sensor proximate the ball of the foot based on the heel breaking contact with the surface at the termination of the stance phase, thereby increasing pressure on the ball of the foot. Conversely or additionally, the step may involve detecting a lack of pressure from heel pressure data detected by the sensor proximate the heel of the foot, given no contact between the heel and the surface at the termination of the stance phase.

The process continues at step 710 by calculating a health status of the knee based on the calculated minimum flexion of the knee at the initiation of the stance phase, the maximum flexion of the knee at the termination of the stance phase, the heel pressure data from the first sensor based on the heel contacting the surface at the initiation of the stance phase, and the ball pressure data detected when the heel breaks contact with the surface at the termination of the stance phase. According to some embodiments, the process can then generate at step 715 an indicator of the health status of the knee in response to the calculated health status of the knee.

Regarding step 710, with reference to FIGS. 7A and 7B, calculating the health status of the knee based on the calculated minimum flexion of the knee at the initiation of the stance phase, the maximum flexion of the knee at the termination of the stance phase, the heel pressure data from the first sensor based on the heel making contact with the surface at the initiation of the stance phase, and the ball pressure data detected when the heel breaks contact with the surface at the termination of the stance phase involves, according to one embodiment, at step 711, comparatively analyzing the calculated minimum and maximum flexion of the knee at the respective initiation and termination of the stance phase for the corresponding leg with a likewise or similarly calculated minimum and maximum flexion of the other knee of the human at the respective initiation and termination of the stance phase for the human's other leg, and at step 712, comparatively analyzing the heel pressure data detected by the first sensor based on the heel contacting with the surface at the initiation of the stance phase, and ball pressure data detected from the ball sensor based on heel breaking contact with the surface at the termination of the stance phase, with heel pressure data detected by a sensor proximate the other heel of the human at the initiation of the stance phase for the corresponding other leg and ball pressure data detected from the ball sensor proximate the ball of the other foot based on the other heel breaking contact with the surface at the termination of the stance phase.

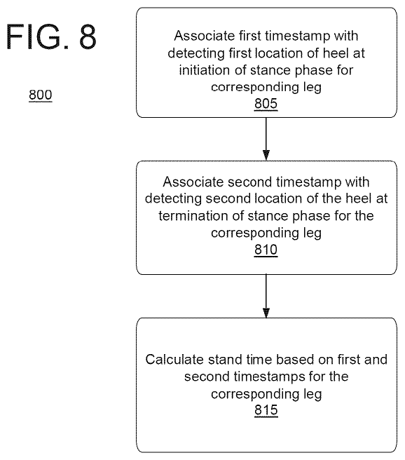

With reference to FIG. 8, according to an embodiment of the invention 800, the system can at step 805 associate a first timestamp with the step 405 of detecting the first location of the heel of the foot of the human from the first sensor proximate the heel at the initiation of the stance phase for the corresponding leg, and at step 810 associate a second timestamp with the step 605 of detecting the second location of the heel from the first sensor at the termination of the stance phase for the corresponding leg. The process continues at step 815 by calculating a “stand time” or “stance time” based on the first and second timestamps for the corresponding leg. This computation provides a measure of strength, endurance, pain tolerance, etc. in the leg based on how long the human takes to complete the stance phase for that leg. This may be particularly insightful when comparing to the stance time for the human's other leg. For example, when one leg is experiencing pain, it is common for a human to place less weight and for a shorter time period on the injured or impaired leg compared, and before shifting weight, to the other, good (pain-free) leg (i.e., before shifting to the stance phase for the other leg).

With reference to FIGS. 10, 11A and 11B, in the same manner as described above in connection with detecting a location of the heel and corresponding knee at the initiation and termination of the stance phase for that leg, it is also possible to detect the location of the heel and corresponding knee at points or locations or times in between the initiation and termination of the stance phase for that leg. For example, embodiments contemplate detecting a second location of the heel from the first sensor at a mid-point 1010 of the stance phase for the corresponding leg, and detecting a second, concurrent, location of the corresponding knee from the second sensor. It is then possible to calculate a second angle θ₂ defined by a vertex, A, at the second location of the knee formed by a first side, B, defined by a straight vertical line that intersects the second location of the knee and a second side, C, defined by a straight line that intersects the second location of the knee and the second location of the heel, and then calculate an amount of flexion of the knee at the mid-point of the stance phase for the corresponding leg based on the second angle θ₂.

Likewise, it is contemplated by embodiments to detect heel pressure data from the first sensor while the heel is in contact with the surface at the mid-point of the stance phase 1010 and to detect ball pressure data from the third sensor while the ball is in contact with the surface at the mid-point of the stance phase 1010. As expected, and depicted by the moderately sized dot at 1012, the first sensor, may, for example, detect a moderate amount of pressure at or on the bottom of the heel when the heel makes contact with the surface at the mid-point of the stance phase. Likewise, as depicted by the moderately sized dot at 1011, the third sensor may, for example, detect a moderate amount of pressure at or on the ball of the foot when the ball contacts the surface at the mid-point of the stand phase 1010.

With the heel and knee location information and the pressure data from the heel and ball sensors, embodiments can calculate a health status of the knee based on the minimum flexion of the knee at the initiation of the stance phase, the amount of flexion of the knee at the mid-point of the stance phase, the heel pressure data detected by the first sensor based on the heel making contact with the surface at the initiation of the stance phase, the heel pressure data detected by the first sensor and the ball pressure data obtained from the third sensor while the heel is in contact with the surface at the mid-point of the stance phase, and generate an indicator of the health status of the knee responsive to the calculated health status of the knee.

In a manner similar to described above with reference to FIGS. 5A, 5B, 7A and 7B, calculating the health status of the knee based on the minimum flexion of the knee at the initiation of the stance phase, the amount of flexion of the knee at the mid-point of the stance phase, the heel pressure data detected by the first sensor based on the heel making contact with the surface at the initiation of the stance phase, the heel pressure data detected by the first sensor and the ball pressure data detected by the third sensor while the heel is in contact with the surface at the mid-point of the stance phase can involve: comparatively analyzing the minimum flexion of the knee at the initiation of the stance phase, the amount of flexion of the knee at the mid-point of the stance phase, and the minimum flexion of the other knee at the initiation of the stance phase and the amount of flexion of the other knee at the mid-point of the stance phase for the corresponding other leg; and comparatively analyzing the heel pressure data detected by the first sensor based on the heel making contact with the surface at the initiation of the stance phase, the heel pressure data detected by the first sensor and the ball pressure data detected by the third sensor while the heel is in contact with the surface at the mid-point of the stance phase for the corresponding leg, heel pressure data detected by a sensor proximate the other heel based on the other heel making contact with the surface at the initiation of the stance phase, and the heel pressure data detected by the first sensor and ball pressure data detected by a sensor proximate the ball of the other foot while the other heel is in contact with the surface at the mid-point of the stance phase for the corresponding other leg.

In yet another embodiment, calculating the health status of the knee may be based on respective locations of heel and knee at the initiation, mid-point, and termination of the stance phase for a corresponding leg. Such an embodiment involves detecting a third location of the heel from the first sensor at a termination of the stance phase for the corresponding leg, detecting a third, concurrent, location of the corresponding knee from the second sensor, and calculating a third angle θ₃ defined by a vertex, A, at the third location of the knee formed by a first side, B, defined by a straight vertical line that intersects the third location of the knee and a second side, C, defined by a straight line that intersects the third location of the knee and the third location of the heel. The embodiment can then calculate a maximum flexion of the knee at the termination of the stance phase for the corresponding leg based on the third angle θ₃.

In addition, the embodiment can detect ball pressure data from the third sensor while the ball is in contact with the surface at the termination of the stance phase, and calculate a health status of the knee based on the minimum flexion of the knee at the initiation of the stance phase, the amount of flexion of the knee at the mid-point of the stance phase, the maximum flexion of the knee at the termination of the stance phase, the heel pressure data detected by the first sensor based on the heel making contact with the surface at the initiation of the stance phase, the heel pressure data detected by the first sensor and the ball pressure data obtained from the third sensor while the heel is in contact with the surface at the mid-point of the stance phase, and the ball pressure data detected by the third sensor while the ball is in contact with the surface at the termination of the stance phase. Based on such, the embodiment can generate an indicator of the health status of the knee responsive to the calculated health status of the knee.

According to the above described embodiment, calculating the health status of the knee based on the minimum flexion of the knee at the initiation of the stance phase, the amount of flexion of the knee at the mid-point of the stance phase, the maximum flexion of the knee at the termination of the stance phase, the heel pressure data detected by the first sensor based on the heel making contact with the surface at the initiation of the stance phase, the heel pressure data detected by the first sensor, the ball pressure data detected by the third sensor while the heel is in contact with the surface at the mid-point of the stance phase, and the ball pressure data detected by the third sensor while the ball is in contact with the surface at the termination of the stance phase, may involve: comparatively analyzing the minimum flexion of the knee at the initiation of the stance phase, the amount of flexion of the knee at the mid-point of the stance phase, the maximum flexion of the knee at the termination of the stance phase, and the minimum flexion of the other knee at the initiation of the stance phase, the amount of flexion of the other knee at the mid-point of the stance phase, the maximum flexion of the other knee at the termination of the stance phase; and comparatively analyzing the heel pressure data detected by the first sensor based on the heel making contact with the surface at the initiation of the stance phase, the heel pressure data detected by the first sensor and the ball pressure data detected by the third sensor while the heel is in contact with the surface at the mid-point of the stance phase, and the ball pressure data detected by the third sensor while the ball is in contact with the surface at the termination of the stance phase, the heel pressure data detected by the first sensor based on the other heel making contact with the surface at the initiation of the stance phase, the heel pressure data detected by the first sensor and the ball pressure data detected by the third sensor while the other heel is in contact with the surface at the mid-point of the stance phase, and the ball pressure data detected by the third sensor while the ball of the other heel is in contact with the surface at the termination of the stance phase.

In the above-described embodiments, calculating the health status of the knee is based on one or more of the respective locations of heel and knee, and corresponding pressure data detected at the initiation, mid-point, and termination of the stance phase for a corresponding leg. It is contemplated that embodiments of the invention may calculate health status of the knee based on one or more of the respective locations of the heel and knee at the initiation, termination, and any point in between the initiation and termination of the stance phase in the same manner as described above. For example, with reference to FIG. 10, it is contemplated that embodiments can detect concurrent knee and heel locations and corresponding pressure data at intermediate phases such as depicted at 1008 and 1012 and similarly calculate the health status of the knee based on such information.

The various sensors described above in connection with the described embodiments may be selected from a wide variety of available sensors depending on their location or placement or orientation on the body or body segment, their functionality or functionalities, cost, size and/or shape, reliability, wearability, durability, ease of set-up and or use, battery life, real-time data gathering and transmission capabilities to support real-time gait analysis, etc. For example, a sensor may comprise an Inertial Measurement Unit (IMU) which combine accelerometers and gyroscopes to measure linear acceleration and angular velocity of the body to which it is attached. For example, an IMU-based sensor placed on the shank (between knee and ankle) and/or foot provide for the ability to obtain heel strike and toe off gait events. Insole pressure sensors are often used in combination with IMU sensors to verify or validate gait events. Other types of sensors, such as mobile or wearable force plate/pressure mat, pressure sensors, magnetometers, may also be used in embodiments of the invention, either stand alone or in combination with the other sensors mentioned herein. Various sensor fusion methods may also be employed to combine information from one or more sensors, for example, to better estimate the data used in the measurements and calculations according to the described embodiments. In some embodiments, sensors may measure kinematics, or geometry of motion, and model these motions mathematically, for example, using algebra.

A pressure sensor, such as an insole pressure sensor, measures the pressure distribution at the foot, which is used to estimate gait parameters such as step count, duration of the gait cycle, swing duration, stance duration, and foot-ground interaction events (such as heel strike or toe off). Such sensors comprise optoelectronic sensors, force-sensing resistors (FSRs), capacitive sensors, and piezoelectric sensors. It is contemplated that a pressure sensor may be combined with an IMU sensor to combine the advantages and functionalities of both types of sensors.

The above referenced electronic computing device may involve and be configured to perform operations, tasks, or features of one or more computers, servers and mobile devices, in accordance with embodiments of the invention. The computing device may be connected to or communicate with one or more external devices and control or perform steps of the methods described herein. The computing device may include, by way of non-limiting examples, server computers, desktop computers, laptop computers, notebook computers, sub-notebook computers, netbook computers, set-top computers, handheld computers, Internet appliances, mobile smartphones, tablet computers, personal digital assistants, embedded computers, wearable computing devices (e.g., AR/VR devices, Google Glass, smart watches, etc.), cloud servers, and any other devices capable of performing calculations. Those of skill in the art will recognize that many mobile or smart phones are suitable for use in the system described herein. Suitable tablet computers include those with booklet, slate, and convertible configurations, known to those of skill in the art.

In various aspects, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, and Windows Server®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In various aspects, the operating system is provided by cloud computing. Those of skill in the art will also recognize that suitable mobile smart phone operating systems include, by way of non-limiting examples, Nokia® Symbian® OS, Apple® iOS®, Google® Android®, Microsoft® Windows Phone® OS, Microsoft® Windows Mobile® OS, and Linux®.

In various aspects, the computing device may include storage. The storage is one or more physical apparatus used to store data or programs on a temporary or permanent basis. In various aspects, the storage may be volatile memory and requires power to maintain stored information. In various aspects, the storage may be non-volatile memory and retains stored information when the computing device is not powered. In various aspects, the non-volatile memory includes flash memory. In various aspects, the non-volatile memory includes dynamic random-access memory (DRAM). In various aspects, the non-volatile memory includes ferroelectric random-access memory (FRAM). In various aspects, the non-volatile memory includes phase-change random access memory (PRAM). In various aspects, the storage includes, by way of non-limiting examples, CD-ROMs, DVDs, flash memory devices, magnetic disk drives, magnetic tapes drives, optical disk drives, solid state drives, and cloud computing-based storage. In various aspects, the storage may be a combination of devices such as those disclosed herein.

The computing device further includes a processor, one or more display devices, an input device, and a network card. The processor executes instructions which implement tasks or functions of programs. When a user executes a program or the program is automatically executed based on a batch process, the processor reads the program stored in the storage, loads the program on the RAM, and executes instructions prescribed by the program.

The processor may include a microprocessor, central processing unit (CPU), application specific integrated circuit (ASIC), arithmetic coprocessor, graphic processor, or image processor, each of which is electronic circuitry within a computer that carries out instructions of one or more computer programs by performing the basic arithmetic, logical, control, and input/output (I/O) operations specified by the instructions.

In embodiments, an extension unit may include several connectors or ports, such as one or more universal serial buses (USBs), parallel ports, and/or expansion slots such as peripheral component interconnect (PCI) and PCI express (PCIe). The extension is not limited to the list but may include other slots or ports that can be used for appropriate purposes. The extension may be used to install hardware or add additional functionalities to a computer that may facilitate the purposes of the computer. For example, a USB port can be used for adding additional storage to the computer.

In various aspects, the display may be a cathode ray tube (CRT), a liquid crystal display (LCD), or light emitting diode (LED). In various aspects, the display may be a thin film transistor liquid crystal display (TFT-LCD). In various aspects, the display may be an organic light emitting diode (OLED) display. In various aspects, the OLED display is a passive-matrix OLED (PMOLED) or active-matrix OLED (AMOLED) display. In various aspects, the display may be a plasma display. In various aspects, the display may be a video projector. In various aspects, the display may be interactive (e.g., having a touch screen or a sensor such as a camera, a 3D sensor, a LiDAR, a radar, etc.) that can detect user interactions/gestures/responses and the like. In still various aspects, the display may be a hologram projector configured to project 3D objects.

A user may input and/or modify data via the input device that may include a keyboard, a mouse, or any other device with which the user may input data. The display may be a touch screen so that the display can be used as the input device.

The network card is used to communicate with other computing devices, wirelessly or via a wired connection. Through the network card, the computing device may receive, modify, and/or update data from and to external devices, including the aforementioned sensors.

Any of the herein described methods or programs may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, C#, Delphi, Fortran, Java, JavaScript, machine code, operating system command languages, Pascal, Perl, PL1, python, scripting languages, Visual Basic, meta-languages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

While several aspects of the disclosure have been shown in the drawings, it is not intended that the disclosure be limited thereto, as it is intended that the disclosure be as broad in scope as the art will allow and that the specification be read likewise. It is to be understood, therefore, that the present disclosure is not limited to the precise aspects described, and that various other changes and modifications may be affected by one skilled in the art without departing from the scope or spirit of the disclosure. Additionally, the elements and features shown and described in connection with certain aspects may be combined with the elements and features of certain other aspects without departing from the scope of the present disclosure, and that such modifications and variation are also included within the scope of the present disclosure. Therefore, the above description should not be construed as limiting, but merely as exemplifications of preferred aspects. Thus, the scope of the aspects should be determined by the appended claims and their legal equivalents, rather than by the examples given.

1. A computer-implemented method for an electronic computing system having a memory to store instructions and a processor to execute the instructions to generate information relating to a gait of a human, comprising:
detecting a first location of a heel of the human from a first sensor coupled in communication with the electronic computing system and proximate the heel at an initiation of a swing phase for a corresponding leg;
detecting a first, concurrent, location of a corresponding knee from a second sensor coupled in communication with the electronic computing system and proximate the knee;
calculating via instructions executed by the processor a first angle θ₁ defined by a vertex, A, at the first location of the knee formed by a first side, B, defined by a straight vertical line that intersects the first location of the knee and a second side, C, defined by a straight line that intersects the first location of the knee and the first location of the heel; and
calculating via instructions executed by the processor a maximum flexion of the knee at the initiation of the swing phase for the corresponding leg based on the first angle θ₁.

2. The computer-implemented method of claim 1, wherein detecting the first location of the heel of the human from the first sensor at the initiation of the swing phase comprises detecting the first location of the heel from the first sensor based on the heel breaking contact with a surface as detected by the first sensor.

3. The computer-implemented method of claim 1, further comprising:
calculating via instructions executed by the processor a plurality of consecutive maximum flexions of the knee at a corresponding plurality of consecutive initiations of the swing phases for the corresponding leg;
associating via instructions executed by the processor a respective timestamp with each of the plurality of consecutive maximum flexions based on the concurrent detections of the respective first locations of the heel and knee; and
generating via instructions executed by the processor an indicator of movement for the human based on a number of the plurality of consecutive maximum flexions with associated respective timestamps that occur during a selected period of time.

4. The computer-implemented method of claim 1, further comprising:
detecting a second location of the heel from the first sensor at a termination of the swing phase for the corresponding leg;
detecting a second, concurrent, location of the knee from the second sensor;
calculating via instructions executed by the processor a second angle θ₂ defined by a vertex, A, at the second location of the knee formed by a first side, B, defined by a straight vertical line that intersects the second location of the knee and a second side, C, defined by a straight line that intersects the second location of the knee and the second location of the heel; and
calculating via instructions executed by the processor a minimum flexion of the knee at the termination of the swing phase based on the second angle θ₂.

5. The computer-implemented method of claim 4, wherein detecting the second location of the heel from the first sensor at the termination of the swing phase comprises detecting the second location of the heel from the first sensor based on the heel making contact with a surface as detected by the first sensor.

6. The computer-implemented method of claim 4, further comprising:
calculating via instructions executed by the processor a third angle θ₃ based on the first angle and the second angle; and
generating via instructions executed by the processor an indicator of an excursion of the knee from the initiation of the swing phase to the termination of the swing phase for the corresponding leg according to the third angle θ₃.

7. The computer-implemented method of claim 6, further comprising:
determining stride length due to motion at the hip of the human, including hip extension stride length and hip flexion stride length, during a swing phase for the corresponding leg; and
determining stride length due to motion at the knee of the human, including both knee flexion stride length and knee extension stride length, during the swing phase for the corresponding leg.

8. The computer-implemented method of claim 7, further comprising:
calculating a maximum knee flexion angle θ₁ based on the knee flexion stride length during the swing phase for the corresponding leg, a length and orientation of a femur for the corresponding leg, and a length and orientation of a tibia in flexion for the corresponding leg; and
calculating a minimum knee flexion angle θ₂ based on the knee extension stride length during the swing phase for the corresponding leg, a length and orientation of a femur for the corresponding leg, and a length and orientation of a tibia in extension for the corresponding leg.

9. A computer-implemented method for an electronic computing system having a memory to store instructions and a processor to execute the instructions to generate information relating to a gait of a human, comprising:
detecting a first location of a heel of a foot of the human from a first sensor coupled in communication with the electronic computing system and proximate the heel at an initiation of a stance phase for a corresponding leg;
detecting a first, concurrent, location of a corresponding knee from a second sensor coupled in communication with the electronic computing system and proximate the knee;
calculating via instructions executed by the processor a first angle θ₁ defined by a vertex, A, at the first location of the knee formed by a first side, B, defined by a straight vertical line that intersects the first location of the knee and a second side, C, defined by a straight line that intersects the first location of the knee and the first location of the heel; and
calculating via instructions executed by the processor a minimum flexion of the knee at the initiation of the stance phase for the corresponding leg based on the first angle θ₁.

10. The computer-implemented method of claim 9, further comprising:
detecting heel pressure data from the first sensor based on the heel making contact with a surface at the initiation of the stance phase;
calculating via instructions executed by the processor a health status of the knee based on the calculated minimum flexion of the knee at the initiation of the stance phase and the heel pressure data from the first sensor based on the heel making contact with the surface at the initiation of the stance phase; and
generating via the instructions executed by the processor an indicator of the health status of the knee responsive to the calculated health status of the knee.

11. The computer-implemented method of claim 10, wherein calculating the health status of the knee based on the calculated minimum flexion of the knee at the initiation of the stance phase and the heel pressure data from the first sensor based on the heel making contact with the surface at the initiation of the stance phase comprises:
comparatively analyzing via instructions executed by the processor the calculated minimum flexion of the knee at the initiation of the stance phase for the corresponding leg and a likewise calculated minimum flexion of another knee of the human at an initiation of a stance phase for a corresponding other leg of the human; and
comparatively analyzing via instructions executed by the processor the heel pressure data detected by the first sensor based on the heel making contact with the surface at the initiation of the stance phase with heel pressure data detected by a sensor proximate another heel of the human at the initiation of the stance phase for the corresponding other leg.

12. The computer-implemented method of claim 9, further comprising:
detecting a second location of the heel from the first sensor at a termination of the stance phase for the corresponding leg;
detecting a second, concurrent, location of the corresponding knee from the second sensor;
calculating via instructions executed by the processor a second angle θ₂ defined by a vertex, A, at the second location of the knee formed by a straight vertical line that intersects the second location of the knee and a second side, C, defined by a straight line that intersects the second location of the knee and the second location of the heel; and
calculating via instructions executed by the process a maximum flexion of the knee at the termination of the stance phase for the corresponding leg based on the second angle θ₂.

13. The computer-implemented method of claim 12, further comprising:
detecting ball pressure data from a third sensor proximate a ball of the foot based on the heel breaking contact with the surface at the termination of the stance phase;
calculating via instructions executed by the processor a health status of the knee based on the calculated minimum flexion of the knee at the initiation of the stance phase, the calculated maximum flexion of the knee at the termination of the stance phase, the heel pressure data detected by the first sensor based on the heel making contact with the surface at the initiation of the stance phase, and the ball pressure data detected by the third sensor based on the heel breaking contact with the surface at the termination of the stance phase; and
generating via the instructions executed by the processor an indicator of the health status of the knee responsive to the calculated health status of the knee.

14. The computer-implemented method of claim 13, wherein calculating via instructions executed by the processor the health status of the knee based on the minimum flexion of the knee at the initiation of the stance phase, the maximum flexion of the knee at the termination of the stance phase, the heel pressure data detected by the first sensor based on the heel making contact with the surface at the initiation of the stance phase, and the ball pressure data detected by the third sensor based on the heel breaking contact with the surface at the termination of the stance phase comprises:
comparatively analyzing via instructions executed by the processor the calculated minimum and maximum flexion of the knee at the respective initiation and termination of the stance phase for the corresponding leg and a likewise calculated minimum and maximum flexion of the other knee at the respective initiation and termination of the stance phase for the corresponding other leg; and
comparatively analyzing via instructions executed by the processor the heel pressure data detected by the first sensor based on the heel making contact with the surface at the initiation of the stance phase, and the ball pressure data detected by the third sensor based on the heel breaking contact with the surface at the termination of the stance phase with heel pressure data detected by a sensor proximate the other heel based on the other heel making contact with the surface at the initiation of the stance phase, and ball pressure data detected by a sensor proximate the ball of the other foot based on the other heel breaking contact with the surface at the termination of the stance phase.

15. The computer-implemented method of claim 12, further comprising:
associating via the instructions executed by the processor a first timestamp with detecting the first location of the heel of the foot of the human from the first sensor proximate the heel at the initiation of the stance phase for the corresponding leg;
associating via the instructions executed by the processor a second timestamp with detecting the second location of the heel from the first sensor at the termination of the stance phase for the corresponding leg; and
calculating via the instructions executed by the processor a stand time based on the first and second timestamps for the corresponding leg.

16. The computer-implemented method of claim 10, further comprising:
detecting a second location of the heel from the first sensor at a mid-point of the stance phase for the corresponding leg;
detecting a second, concurrent, location of the corresponding knee from the second sensor;
calculating via instructions executed by the processor a second angle θ₂ defined by a vertex, A, at the second location of the knee formed by a first side, B, defined by a straight vertical line that intersects the second location of the knee and a second side, C, defined by a straight line that intersects the second location of the knee and the second location of the heel; and
calculating via the instructions executed by the processor an amount of flexion of the knee at the mid-point of the stance phase for the corresponding leg based on the second angle θ₂.

17. The computer-implemented method of claim 16, further comprising:
detecting heel pressure data from the first sensor while the heel is in contact with the surface at the mid-point of the stance phase; and
detecting ball pressure data from the third sensor while the ball is in contact with the surface at the mid-point of the stance phase;
calculating via the instructions executed by the processor a health status of the knee based on the minimum flexion of the knee at the initiation of the stance phase, the amount of flexion of the knee at the mid-point of the stance phase, the heel pressure data detected by the first sensor based on the heel making contact with the surface at the initiation of the stance phase, the heel pressure data detected by the first sensor and the ball pressure data obtained from the third sensor while the heel is in contact with the surface at the mid-point of the stance phase; and
generating via the instructions executed by the processor an indicator of the health status of the knee responsive to the calculated health status of the knee.

18. The computer-implemented method of claim 17, wherein calculating the health status of the knee based on the minimum flexion of the knee at the initiation of the stance phase, the amount of flexion of the knee at the mid-point of the stance phase, the heel pressure data detected by the first sensor based on the heel making contact with the surface at the initiation of the stance phase, the heel pressure data detected by the first sensor and the ball pressure data detected by the third sensor while the heel is in contact with the surface at the mid-point of the stance phase, comprises:
comparatively analyzing via instructions executed by the processor the minimum flexion of the knee at the initiation of the stance phase, the amount of flexion of the knee at the mid-point of the stance phase, and the minimum flexion of the other knee at the initiation of the stance phase and the amount of flexion of the other knee at the mid-point of the stance phase for the corresponding other leg; and
comparatively analyzing via instructions executed by the processor the heel pressure data detected by the first sensor based on the heel making contact with the surface at the initiation of the stance phase, the heel pressure data detected by the first sensor and the ball pressure data detected by the third sensor while the heel is in contact with the surface at the mid-point of the stance phase for the corresponding leg, heel pressure data detected by a sensor proximate the other heel based on the other heel making contact with the surface at the initiation of the stance phase, and the heel pressure data detected by the first sensor and ball pressure data detected by a sensor proximate the ball of the other foot while the other heel is in contact with the surface at the mid-point of the stance phase for the corresponding other leg.

19. The computer-implemented method of claim 17, further comprising:
detecting a third location of the heel from the first sensor at a termination of the stance phase for the corresponding leg;
detecting a third, concurrent, location of the corresponding knee from the second sensor;
calculating via instructions executed by the processor a third angle θ₃ defined by a vertex, A, at the third location of the knee formed by a first side, B, defined by a straight vertical line that intersects the third location of the knee and a second side, C, defined by a straight line that intersects the third location of the knee and the third location of the heel; and
calculating via instructions executed by the processor a maximum flexion of the knee at the termination of the stance phase for the corresponding leg based on the third angle θ₃.

20. The computer-implemented method of claim 19, further comprising:
detecting ball pressure data from the third sensor while the ball is in contact with the surface at the termination of the stance phase;
calculating a health status of the knee based on the minimum flexion of the knee at the initiation of the stance phase, the amount of flexion of the knee at the mid-point of the stance phase, the maximum flexion of the knee at the termination of the stance phase, the heel pressure data detected by the first sensor based on the heel making contact with the surface at the initiation of the stance phase, the heel pressure data detected by the first sensor and the ball pressure data obtained from the third sensor while the heel is in contact with the surface at the mid-point of the stance phase, and the ball pressure data detected by the third sensor while the ball is in contact with the surface at the termination of the stance phase; and
generating via the instructions executed by the processor an indicator of the health status of the knee responsive to the calculated health status of the knee.

21. The computer-implemented method of claim 20, wherein calculating the health status of the knee based on the minimum flexion of the knee at the initiation of the stance phase, the amount of flexion of the knee at the mid-point of the stance phase, the maximum flexion of the knee at the termination of the stance phase, the heel pressure data detected by the first sensor based on the heel making contact with the surface at the initiation of the stance phase, the heel pressure data detected by the first sensor and the ball pressure data detected by the third sensor while the heel is in contact with the surface at the mid-point of the stance phase, and the ball pressure data detected by the third sensor while the ball is in contact with the surface at the termination of the stance phase, comprises:
comparatively analyzing the minimum flexion of the knee at the initiation of the stance phase, the amount of flexion of the knee at the mid-point of the stance phase, the maximum flexion of the knee at the termination of the stance phase, and the minimum flexion of the other knee at the initiation of the stance phase, the amount of flexion of the other knee at the mid-point of the stance phase, the maximum flexion of the other knee at the termination of the stance phase; and
comparatively analyzing the heel pressure data detected by the first sensor based on the heel making contact with the surface at the initiation of the stance phase, the heel pressure data detected by the first sensor and the ball pressure data detected by the third sensor while the heel is in contact with the surface at the mid-point of the stance phase, and the ball pressure data detected by the third sensor while the ball is in contact with the surface at the termination of the stance phase, the heel pressure data detected by the first sensor based on the other heel making contact with the surface at the initiation of the stance phase, the heel pressure data detected by the first sensor and the ball pressure data detected by the third sensor while the other heel is in contact with the surface at the mid-point of the stance phase, and the ball pressure data detected by the third sensor while the ball of the other heel is in contact with the surface at the termination of the stance phase.
